SEQUENCE LISTING (4 sequences)

SEQ ID NO: 1 - DNA, 34 bp, synthetic construct
  source 1..34: mol_type = other DNA
gtttgcggccgcaagagaggtgagaaccatcaag

SEQ ID NO: 2 - DNA, 35 bp, synthetic construct
  source 1..35: mol_type = other DNA
ggtgtctagattaaggttttatggttgtggtatag

SEQ ID NO: 3 - DNA, 34 bp, synthetic construct
  source 1..34: mol_type = other DNA
gtttaagcttgccaccatggcttccaaggtgtac

SEQ ID NO: 4 - DNA, 31 bp, synthetic construct
  source 1..31: mol_type = other DNA
tttgctcgagtcactgctcgttcttcagcac

(19) United States
(12) Patent Application Publication
(10) Pub. No.: US 2023/0381141 A1
(43) Pub. Date: Nov. 30, 2023

(54) CONJOINT THERAPIES FOR TREATING VIRAL INFECTIONS

(71) Applicant: The Regents of the University of California, Oakland, CA (US)

(72) Inventors: Varghese John, Los Angeles, CA (US); Robert Damoiseaux, Los Angeles, CA (US); Melody Li, Los Angeles, CA (US); Barbara Jagodzinska, Redwood City, CA (US); Jesus J. Campagna, Playa Del Rey, CA (US); Constance Yuen, Los Angeles, CA (US); Pablo Alvarez, Los Angeles, CA (US)

(21) Appl. No.: 18/178,266

(22) Filed: Mar. 3, 2023

Related U.S. Application Data
(60) Provisional application No. 63/316,658, filed on Mar. 4, 2022.

(51) Int. Cl. A61K 31/4152; A61K 31/404; A61K 31/403; A61K 31/427

(52) U.S. Cl. CPC A61K 31/4152; A61K 31/404; A61K 31/403; A61K 31/427

(57) ABSTRACT

The present disclosure relates to methods of treating coronavirus diseases comprising conjointly administering a combination of a PLpro inhibitor and an Mpro inhibitor, and to associated compositions.

### CROSS-REFRENCE TO RELATED APPLICATIONS

This application claims the benefit of, and priority to, U.S. Patent Application No. 63/316,658, filed Mar. 4, 2022, the entire content of which is incorporated by reference herein in its entirety.

### SEQUENCE LISTING

The instant application contains a Sequence Listing which has been submitted electronically in XML file format and is hereby incorporated by reference in its entirety. Said XML copy, created on Jun. 23, 2023, is named UCH-32401_SL.xml and is 4,621 bytes in size.

### BACKGROUND

Coronavirus Infectious Disease-19 (COVID-19) is an infectious disease caused by the SARS-CoV-2 virus. For older people and people with underlying health issues, the disease can lead to life-threatening inflammatory responses, with lung damage and subsequent lung failure. SARS-CoV-2 requires two cysteine proteases, the main protease (Mpro) and the papain-like protease (PLpro) that are essential for polypeptide processing during viral maturation and replication. PLpro also removes post-translational modifications by ubiquitin (Ub) and interferon-stimulated gene product 15 (ISG15) from host proteins through its deubiquitinase domain, leading to the ability of the virus to evade the host anti-viral immune response.

### SUMMARY OF THE INVENTION

The present disclosure provides methods of treating a coronavirus infection, such as SARS-CoV-2, comprising conjointly administering to a subject in need thereof:

a PLpro inhibitor, or a pharmaceutically acceptable salt or prodrug thereof; and

an Mpro inhibitor, or a pharmaceutically acceptable salt or prodrug thereof.

In some embodiments, the PLpro inhibitor is:

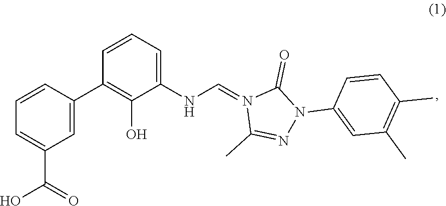

or a pharmaceutically acceptable salt thereof. In other embodiments, the PLpro inhibitor is:

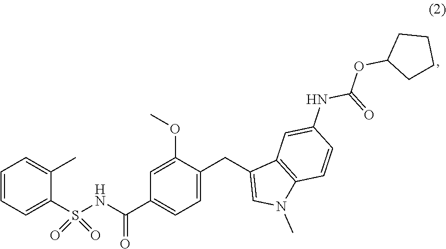 [SMILES: COc1cc(C(=O)NS(=O)(=O)c2ccccc2C)ccc1Cc1cn(C)c2ccc(NC(=O)OC3CCCC3)cc12]

or a pharmaceutically acceptable salt thereof.

In some embodiments, the Mpro inhibitor is:

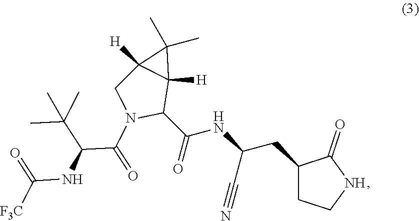 [SMILES: CC(C)(C)[C@H](NC(=O)C(F)(F)F)C(=O)N1C[C@H]2[C@@H](C1C(=O)N[C@H](C#N)C[C@@H]1CCNC1=O)C2(C)C]

or a pharmaceutically acceptable salt thereof.

In some embodiments, the method further comprises administering ritonavir:

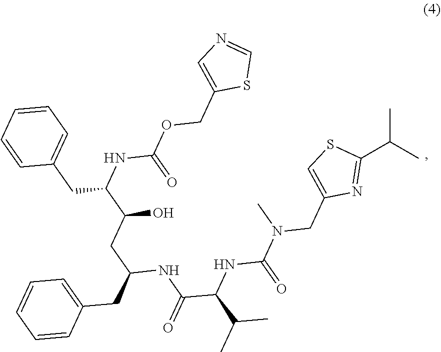 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

or a pharmaceutically acceptable salt thereof.

In some embodiments, the steady state plasma Cₘₐₓ of the PLpro inhibitor is at least about 10 μM, e.g., from about 10 μM to about 100 μM, or from about 40 μM to about 60 μM.

In some embodiments, the PLpro inhibitor and/or the Mpro inhibitor is administered orally.

In some embodiments, the PLpro inhibitor and/or the Mpro inhibitor is administered daily. In certain embodiments, the PLpro inhibitor and Mpro inhibitor are administered concomitantly. In other embodiments, the PLpro inhibitor and Mpro inhibitor are administered sequentially.

In some embodiments, the PLpro inhibitor and the Mpro inhibitor are administered in separate dosage forms. For example, the PLpro inhibitor may be administered in a first pharmaceutical composition, e.g., a solid dosage form that comprises one or more pharmaceutically acceptable excipients and the Mpro inhibitor may be administered in a second pharmaceutical composition, e.g., a solid dosage form that comprises one or more pharmaceutically acceptable excipients. In some embodiments, the first composition comprises between 50-100 mg of the PLpro inhibitor, preferably about 75 mg of the PLpro inhibitor. In other embodiments, the PLpro inhibitor and the Mpro inhibitor are administered in a single dosage form.

In certain embodiments, the present disclosure provides a pharmaceutical composition comprising:

a PLpro inhibitor, or a pharmaceutically acceptable salt or prodrug thereof;

an Mpro inhibitor, or a pharmaceutically acceptable salt or prodrug thereof; and

one or more pharmaceutically acceptable excipients.

In certain embodiments, the PLpro inhibitor is:

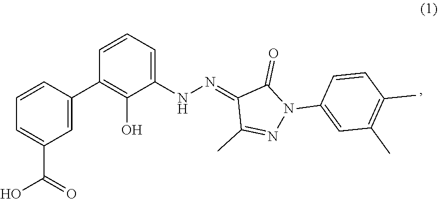 [SMILES: CC1=NN(c2ccc(C)c(C)c2)C(=O)/C1=N/Nc1cccc(-c2cccc(C(=O)O)c2)c1O]

or a pharmaceutically acceptable salt thereof. In other embodiments, the PLpro inhibitor is:

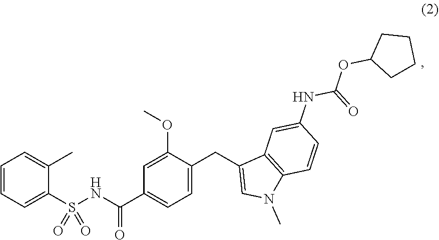 [SMILES: COc1cc(C(=O)NS(=O)(=O)c2ccccc2C)ccc1Cc1cn(C)c2ccc(NC(=O)OC3CCCC3)cc12]

or a pharmaceutically acceptable salt thereof.

In certain embodiments, the Mpro inhibitor is:

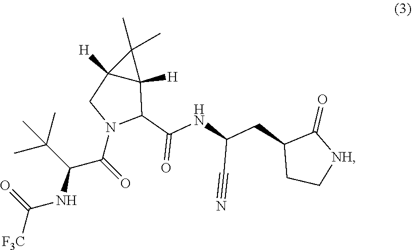 [SMILES: CC(C)(C)[C@H](NC(=O)C(F)(F)F)C(=O)N1C[C@H]2[C@@H](C1C(=O)N[C@H](C#N)C[C@@H]1CCNC1=O)C2(C)C]

or a pharmaceutically acceptable salt thereof.

In certain embodiments, the pharmaceutical composition further comprises ritonavir:

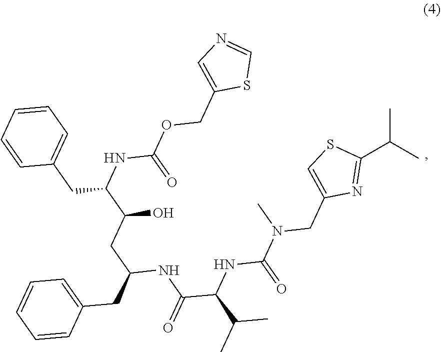 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

or a pharmaceutically acceptable salt thereof.

### BRIEF DESCRIPTION OF THE DRAWINGS

FIG. 1. DDL-701 dose response in the three assays: deubiquitinase, PLpro and ISG15, showing an IC₅₀ 21, 13 and 8 μM respectively.

FIG. 2. DDL-701 in combination with DDL-750 or DDL-750/DDL-751 inhibits PLpro and its deubiquitinase and ISG15 activities in the three assays.

FIG. 3. DDL-701 antiviral and cell viability assays.

FIG. 4. Interferon induction assay.

FIG. 5. Plasma drug levels 2 hours post oral administration.

FIG. 6. Scattergraph of clinical compound library screening using the PL^{pro} assay.

FIGS. 7A-7B. DDL-701 and DDL-750 alone and in combination in the PL^{pro} and M^{pro} assays. FIG. 7A. PL^{pro} assays with DDL-701 and DDL-750 each alone and in combination at the concentrations shown. Statistical analysis performed using one-way ANOVA with post-hoc comparison of each treated well to the control, where ****p<0.0001. FIG. 7B. M^{pro} assays with DDL-701 and DDL-750 each alone and in combination at the concentrations shown. Statistical analysis performed using one-way ANOVA with post-hoc comparison of each treated well to the control, where ****p<0.0001.

FIG. 8. DDL-701 partially restores interferon-beta (IFN-b) induction by poly I:C in the presence of PL^{pro}. Polyinosinic:polycytidylic acid (poly I:C) was used to stimulate IFN-β induction in HEK-293 cells. Transfection with a reporter construct expressing Wuhan strain PL^{pro} (see legend in table) significantly decreases IFN-β induction as compared to the poly I:C only control, which is partially restored by the PL^{pro} inhibitor DDL-701, but not by DDL-715 or M^{pro} inhibitor DDL-750, all at 1 μM. Statistical analysis performed using one-way ANOVA with Dunnett's post-hoc comparison of the poly I:C only control to all other groups and Sidak' s post-hoc comparison of poly I:C+PL^{pro} no drug (--) to Poly I:C+PL^{pro}+DDL-701, where ****p<0.0001 and *p≤0.05.

FIG. 9. Plasma and brain levels of DDL-701 and DDL-750 in mice. Mice received either DDL-701 or 750 alone or in combination by oral dosing at 30 mg/Kg. Plasma (left) and brain (right) levels were assessed 2 hours after dosing. DDL-701 n=3, DDL-750 n=1, and DDL-701+750 n=2.

FIG. 10. Putative restoration of host immune responses by a DDL-701/nirmatrelvir cocktail. PL^{pro} alters IRF3 phosphorylation activity and the IFN-β host response. In combination with an M^{pro} inhibitor such as nirmatrelvir, DDL-701 could help restore the host cell immune response in addition to reducing viral replication.

### DETAILED DESCRIPTION OF THE INVENTION

The novel coronavirus, named severe acute respiratory syndrome coronavirus 2 (SARS-CoV-2), requires two cysteine proteases, the main protease (Mpro) and the papain-like protease (PLpro), that are essential for polypeptide processing during viral maturation and replication. PLpro also removes post-translational modifications by ubiquitin (Ub) and interferon-stimulated gene product 15 (ISG15) from host proteins through its deubiquitinase domain, leading to the ability of the virus to evade the host anti-viral immune response. PLpro, in this virus, has been reported to mediate cleavage of ISG15 from interferon regulatory factor 3 (IRF3), blocking its nuclear translocation, and reducing type 1 interferon responses. PLpro is a promising therapeutic target. Pharmacological inhibition of its protease and deubiquitinase activities can block polyprotein processing and viral replication, and promote anti-viral immunity reducing viral release from infected cells, respectively. Through virtual & real-time screening of an FDA compound library two currently used drug candidates have been identified that inhibit PLpro. DDL-701 and DDL-715 (structures given below) inhibit the PLpro enzyme either by themselves or in combination with the Pfizer Mpro inhibitor nirmatrelvir (DDL-750) and ritonavir (DDL-751), which are a component of the approved Paxlovid treatment for COVID infection.

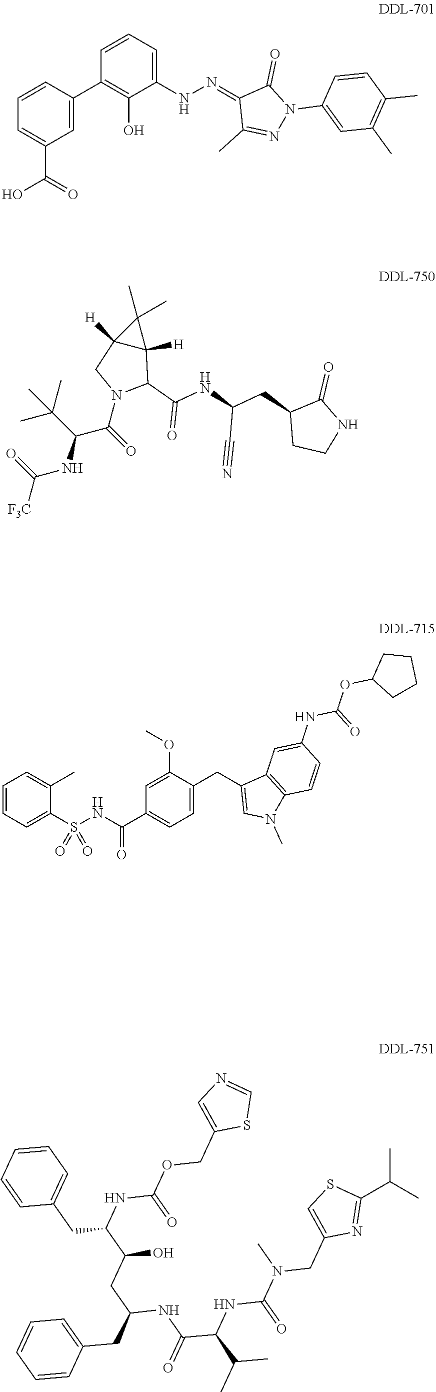 [SMILES: CC1=NN(c2ccc(C)c(C)c2)C(=O)/C1=N/Nc1cccc(-c2cccc(C(=O)O)c2)c1O ; CC(C)(C)[C@H](NC(=O)C(F)(F)F)C(=O)N1C[C@H]2[C@@H](C1C(=O)N[C@H](C#N)C[C@@H]1CCNC1=O)C2(C)C ; COc1cc(C(=O)NS(=O)(=O)c2ccccc2C)ccc1Cc1cn(C)c2ccc(NC(=O)OC3CCCC3)cc12 ; CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

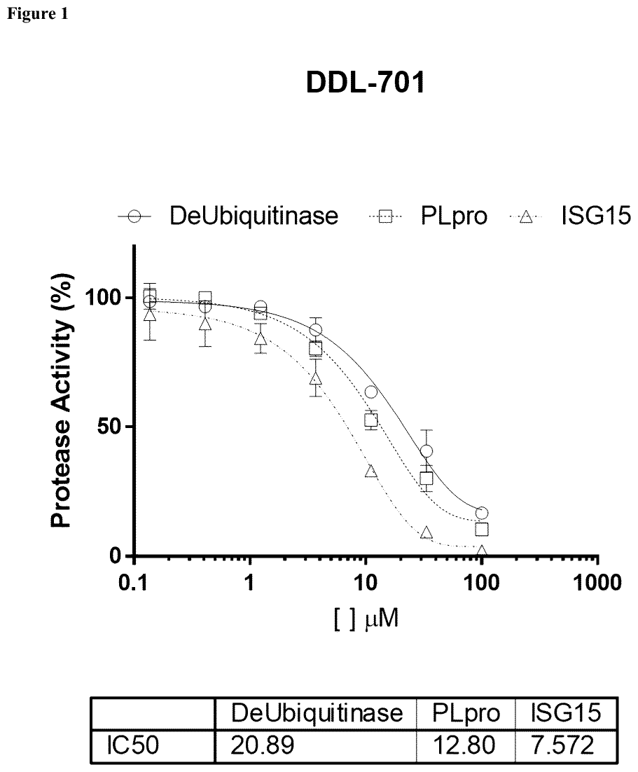

Through screening, it has been identified that a clinically used drug (DDL-701) is a PLpro inhibitor. Importantly DDL-701 is currently FDA approved for human oral dosing. DDL-701 is currently used as a 75 mg tablet and after oral dosing the steady state drug level reached in plasma is Cₘₐₓ ˜50 uM. At this dose DDL-701 inhibits PLpro and suppress its deubiquitinase and ISG15 cleavage activities (FIG. 1).

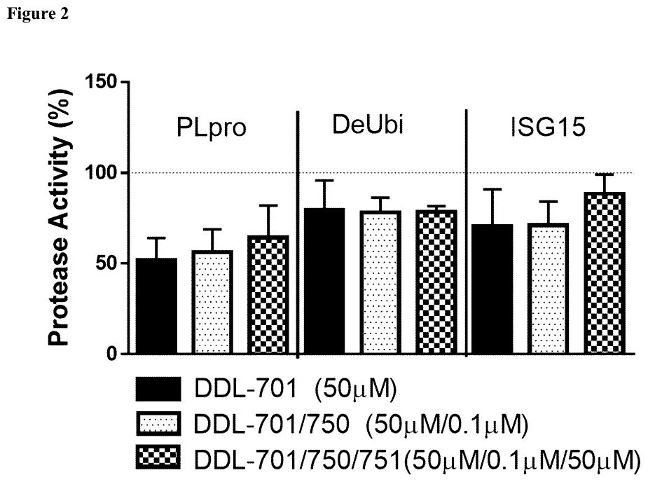

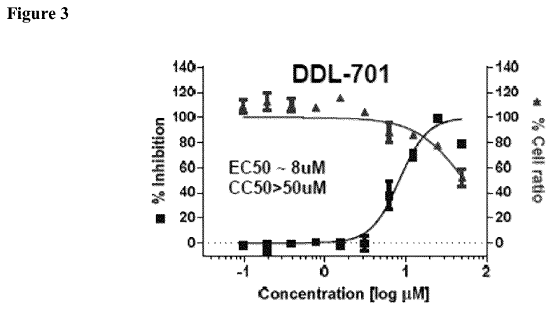

In combination with the protease inhibitor nirmatrelvir (depicted in FIG. 2 as DDL-750) or with both nirmatrelvir and ritonavir (depicted in FIG. 2 as DDL-751), which together are the components of Paxlovid, DDL-701 continues to show its inhibition of PLpro (FIG. 2). DDL-701 also has been reported (Sangeun J. et al., 2020) to show robust antiviral activity in Vero cells infected with SARS-CoV-2, with an EC50 of ˜8 uM (FIG. 3). Again, after ingestion of a single 75 mg tablet of DDL-701 plasma drug levels reached are well above the observed EC50 for antiviral activity.

Based upon preliminary data, it is anticipated that DDL-701, through its inhibition of PLpro and in combination with Paxlovid, should inhibit both Mpro and PLpro proteases, leading to robust suppression of the COVID infection.

DDL-701 can be taken orally as a 75 mg tablet (Promacta from Novartis) alone or in combination with the currently approved protease drug Paxlovid that received an Emergency Use Authorization status for COVID-19 treatment. The combination would be expected to inhibit both proteases Mpro and PLpro of the COVID-19 virus and should produce robust suppression of the infection if taken within a few days of testing positive. This drug combination is expected to be equally effective against all the known current viral variants.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds disclosed herein for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

“Prodrug” or “pharmaceutically acceptable prodrug” refers to a compound that is metabolized, for example hydrolyzed or oxidized, in the host after administration to form the compound of the present disclosure. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples which are included merely for purposes of illustration of certain aspects and embodiments of the present invention, and are not intended to limit the invention.

### Methods

### IFN Induction Assays

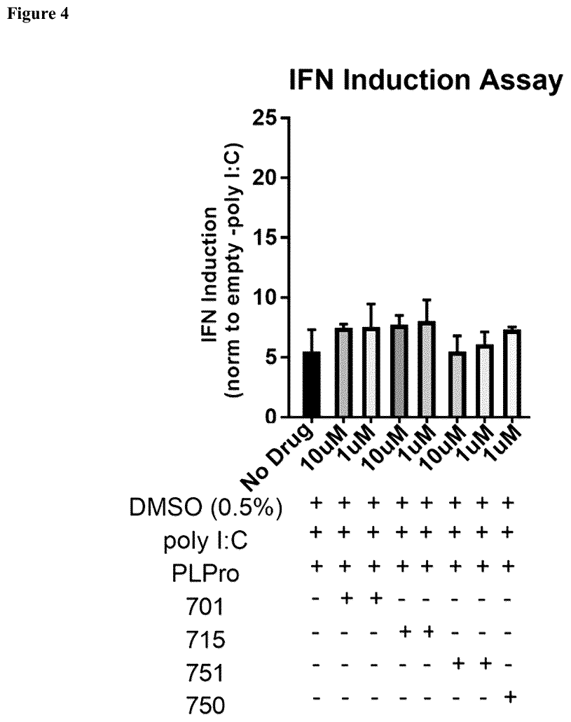

Cells were transfected with a plasmid construct in which a promoter region containing 2 tandem repeats of the transcription factor IRF3 binding sites drives the expression of Firefly luciferase, followed by poly(I:C) transfection and luciferase assay (FIG. 4). It was found that transfection with 50 ng of reporter construct and 500 ng of poly(I:C) is optimal. This condition provides a ˜1-log dynamic range between specific and background signals where we are able to detect inhibition of IFNB response by SARS-CoV-2 nsP3 and potential blocking of that inhibitory activity by candidate compounds. It was determined that: (a) DDL-701 and DDL-715 at 10 and 1 μM produced IFN induction of approximately 1.5 fold over DMSO; (b) DDL-751 did not produce IFN induction at the concentration tested; and (c) DDL-750 at 1 μM produced IFN induction of approximately 1.3 fold over DMSO.

PL^{pro}, M^{pro}, Deubiquitinase, and ISG15 in Vitro Assays

PL^{pro} and PL^{pro} deubiquitinase substrate kits each containing both the enzyme and substrate were purchased from BPS Bioscience (cat #79995-2 & cat #79996 respectively), and for the PL^{pro} ISG15 substrate assay, rhodamine 110 was purchased from South Bay Bio (cat #SBB-PS0002), this kit also contains the PL^{pro} enzyme and ISG15 substrate; each assay was performed following the manufacturer's recommendations, in a 384-well plate format. For the M^{pro} assay, the M^{pro} enzyme was obtained from the O'Donoghue lab at UCSD, the fluorogenic substrate for SARS-CoV-2 M^{pro} was purchased from Vivitide (cat #SFP-3250-v).

Briefly, in each assay, the enzyme was loaded into each well in the appropriate buffer, next the compounds were added into the well and incubated for 10-60 min at 37° C. The reaction was initiated by addition of substrate to each well and the fluorescent signal was read for 60 min at the appropriate excitation/emission.

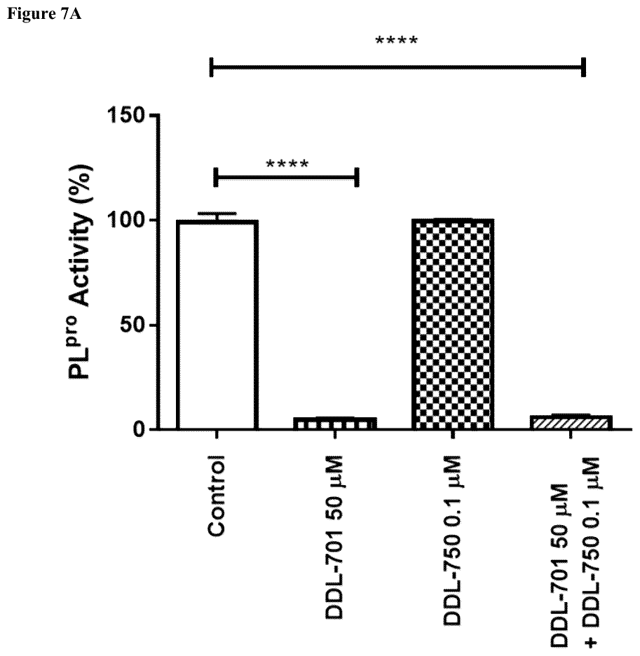

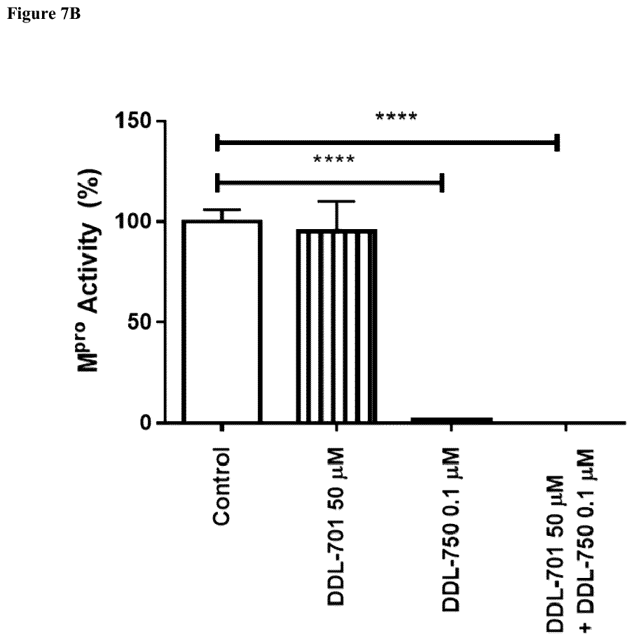

For the PL^{pro} and PL^{pro} deubiquitinase assays, DTT was added to the assay buffer to achieve 1 mM DTT before the assay. A 10 mM stock of compounds was prepared in DMSO, this stock was diluted in assay buffer to achieve a 10× of the desire concentration in the well. Next, the PL^{pro} and deubiquitinase substrates were diluted to 42 μM and 0.5 μM respectively in the assay buffer. Then, PL^{pro} enzyme was diluted to 2.5 ng/μL and 6 ng/μL for the PL^{pro} and deubiquitinase assays respectively in the assay buffer. The reaction was loading 2.5 μL of compounds to each well, then 10 μL of PL^{pro} enzyme was added, follow by an incubation of 1 hours. The reaction was started by adding 12.5 μL of the substrate to each well, the reaction was monitored for 1 h at excitation at 360 nm; emission at 460 nm. For the ISG15 substrate, PL^{pro} enzyme at 0.1 nM was incubated with compounds for lh and the reaction was initiated by adding ISG15-RH110 substrate at 500 nM, the reaction was monitored for 1-hour excitation at 485 nm; emission at 535 nm. For the scatterplot analysis for PL^{pro} enzyme inhibitors from the custom clinical library compounds, treatment with compounds was done at 50 μM. For the PL^{pro} enzyme inhibition in FIG. 7A, DDL-701 was tested at 50 μM, DDL-750 (nirmatrelvir, MedChemExpress) at 0.1 μM and the combination of DDL-701/750 was tested at 50 μM/0.1 μM respectively. For the M^{pro} activity assay, 8 μL of M^{pro} at 5 μg/mL in assay buffer was loaded into each well, followed by 100 nL of the compounds at 50 μM and incubated for 10 min at 37° C. Following the incubation, 2 μL of substrate at 25 μM was added into the wells and the fluorescent signal (ex/em 380/455 nm) was recorded for 1 h at 37° C.

### Cloning of Expression and Reporter Constructs

The SARS CoV-2 papain-like protease (PL^{pro}) domain of Nsp3 was cloned from a doxycycline-inducible piggyBac transposon vector (PB-TAC-ERP2, Addgene #80478) containing the synthesized full-length Nsp3 from the Wuhan-Hu-1 SARS CoV 2 strain (Alvarez and Yao, unpublished). The PL^{pro} domain (amino acids 745-1061) was cloned using PCR (Forward primer 5′-GTTTGCGGCCGCAAGAGAGGTGAGAACCATCAAG-3′ (SEQ ID NO: 1), Reverse primer 5′-GGTGTCTAGATTAAGGTTTTATGGTTGTGGTATAG-3′ (SEQ ID NO: 2) and inserted into myc-pcDNA3.1 using NotI and XbaI restriction sites. For the Renilla luciferase reporter construct, the Renilla luciferase gene was amplified from a Japanese encephalitis virus replicon containing a Renilla luciferase reporter gene using PCR (Forward primer 5′-GTTTAAGCTTGCCACCATGGCTTCCAAGGTGTAC-3′ (SEQ ID NO: 3), Reverse primer 5′-TTTGCTCGAGTCACTGCTCGTTCTTCAGCAC-3′ (SEQ ID NO: 4) and inserted into V5-pcDNA3.1 using HindIII and XhoI restriction sites, removing the V5 tag. An Addgene plasmid was used for the IFN-β promoter luciferase reporter (IFN-Beta_pGL3, #102597). To make the NF-κB signaling reporter, a gene block containing 5 tandem consensus NF-κB binding sites (Badr et al 2009; Ngo et al 2020), followed by the -55 to +19 region of the human IFN-β gene (UCSC Genome Browser) was synthesized by Integrated DNA Technologies (Alvarez and Yao, unpublished). The gene block was then moved into the pCR™-Blunt II-TOPO™ vector using the Zero Blunt™ TOPO™ PCR Cloning Kit (ThermoFisher) and using EcoRI and NheI to move the gene block into IFN-Beta_pGL3, replacing the IFN-β promoter.

For SARS CoV-2 M^{pro}, the plasmid was provided by Rolf Hilgenfeld, University of Lubeck, Germany and transformed into Escherichia coli strain BL21-Gold (DE3). The expression and purification of the protein has been described in detail previously

### IFN-β Induction and NFκB Signaling Luciferase Reporter Assays

To assess whether the compounds could rescue IFN-β induction, HEK293T cells were treated with compound by replacing the media with new media containing the compound at the desired concentration. The cells were then co-transfected with 150 ng of an IFN-β promoter-Firefly luciferase reporter plasmid, 10 ng of a Renilla luciferase expression plasmid, and 250 ng of either an empty myc-pcDNA3.1 vector or a plasmid expressing SARS-CoV-2 PL^{pro} domain of Nsp3 (amino acids 745-1061), using the TransIT-mRNA Transfection Kit (Mirus bio). To induce IFN-β, 500 ng of poly I:C (InvivoGen) was added into the co-transfection mix.

For the NFκB signaling assay, cells were co-transfected with 150 ng of an NFκB dependent-Firefly luciferase reporter plasmid wherein the luciferase gene expression was under control of 5 tandem NFκB binding sites followed by the −55 to +19 region of the human IFNB1 gene for correct spacing, 10 ng of a Renilla luciferase expression plasmid, and 250 ng of either an empty myc-pcDNA3.1 vector or a plasmid expressing SARS-CoV-2 PL^{pro} domain of Nsp3 (amino acids 745-1061), using the TransIT-mRNA Transfection Kit (Mirus bio). To stimulate signaling, cell media was replaced with media containing the compound and 20 ng/mL tumor necrosis factor alpha TNF-α (R&D Systems).

For both assays, cells were harvested 24 hours post transfection using 1× passive lysis buffer and the dual luciferase assay was performed according to instructions from manufacturer (Promega). Luciferase signals were read using a BioTek Synergy H1 plate reader (Thermo Fisher Scientific).

### Pharmacokinetics Analysis in Mice

### Plasma Drug Level Determination

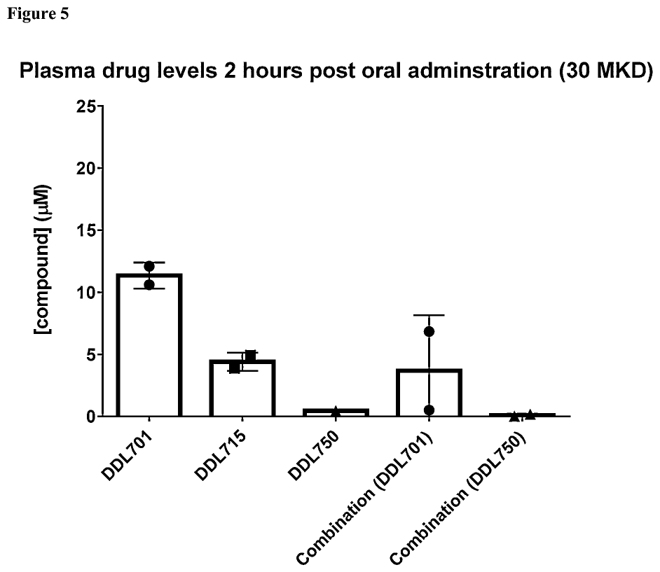

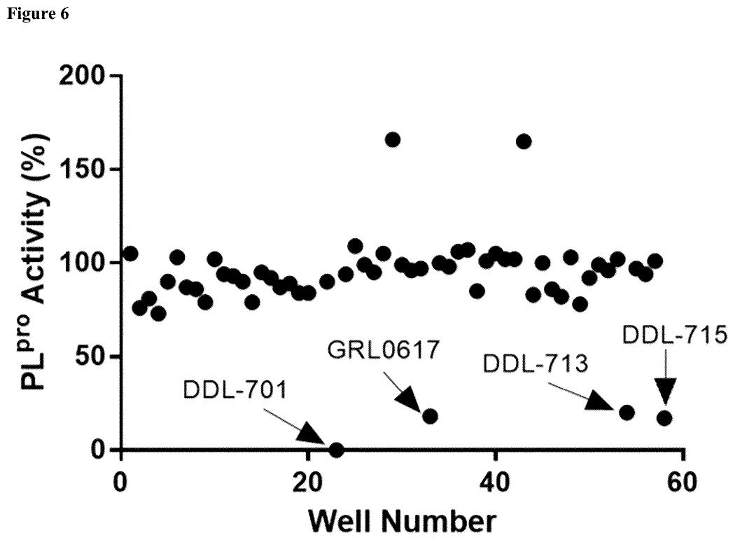

Mice were dosed with: DDL-701, DDL-715, DDL-750 and a combination of DDL-701+DDL-750 by oral gavage at 30 mg/kg. Following compound administration, mice were euthanized at 2 hours, followed by perfusion. Plasma levels were assessed in a targeted liquid chromatography-tandem mass spectrometry (LC-MS/MS) (FIG. 5). Plasma levels for DDL-701, DDL-715 and DDL-750 were approximately 11 μM, 4 μM and 0.4 μM respectively, while for the combination of DDL-701+DDL-750 the plasma levels were 4 μM for DDL-701 and 0.1 μM for DDL-750.

Thus, adult C57BL/6 male mice received DDL-701 or DDL-750 in DMSO alone or in combination at 30 mg/Kg (each) by oral gavage. Two hours after dosing, mice were anesthetized and blood was collected following transcardial puncture for the isolation of plasma. Mice were then perfused transcardially with saline and brain tissue removed. Levels of DDL-701 and DDL-750 were determined in brain and plasma by liquid chromatography-tandem mass spectrometry (LC-MS/MS). Briefly, a targeted LC-MS/MS assay was developed for each compound using the multiple reaction monitoring (MRM) acquisition method on a 6460 triple quadrupole mass spectrometer (Agilent Technologies) coupled to a 1290 Infinity HPLC system (Agilent Technologies) with a Phenomenex analytical column (Kinetex 1.7 μm C18 100 Å 100×2.1 mm). The HPLC method utilized a mixture of solvent A (99.9/1 Water/Formic Acid) and solvent B (99.9/1 Acetonitrile/Formic Acid) and a gradient was use for the elution of the compounds (min/% B: 0/20, 3/20, 19/99, 20/99, 21/20, 35/20). In this assay, detection of fragmented ions originating from each compound at specific chromatographic retention times were utilized to ensure specificity and accurate quantification in the complex biological samples (DDL-701 (M+H)⁺: 443.1, fragment ions: 211.0/322.1, retention time: 21.6; DDL-750 (M+H)⁺: 500.2, fragment ions: 111.0/319.1, retention time: 9.1). Chromatographic peak areas from standards made in drug naïve plasma and brain tissue lysates with increasing amounts of DDL-701 and DDL-750 were used to make a standard curve, and the trendline equation was used to calculate the absolute concentrations of each compound.

### Modeling of Compound Docking to the PL^{pro} Active Site

The crystal structure of PL^{pro} protein (PDB ID: 7CMD) with GRL-0617 ligand was used as a basis for docking studies. The binding site was defined by placing GRL-0617 in the center of the grid box of approximately 20 Å dimensions.

Flare (Cresset Software, v5.0.0) was used for docking modeling. The protein was prepared for docking utilizing an internal optimization module. Three separate docking runs were performed for each compound, each generating unbiased ˜200 poses, and each set of poses was evaluated for the best fit. For the final manual expert evaluation, ˜5-10 poses were selected and then further narrowed to 1-2 poses with the best scores. Each final pose was then examined to identify likely interactions with protein residues (hydrogen bonding, π-π or hydrophobic interactions, etc.). Orientations of compounds showed strong structural interaction with Tyr268 that lies outside the active site tunnel and were considered as preferred binding site of these molecules through π-π and hydrogen bonding interaction with Tyr268. Such compound binding interaction could interfere with PL^{pro} substrate binding and proteolytic activity.

### Results

### Screening of Clinical Compound Library in PL^{pro} Assay Reveals Hit DDL-701

Small molecules from a 58-compound custom clinical library that was assembled from commercial sources were screened for their ability to inhibit PL^{pro} enzyme activity using a short peptide substrate. As shown in the scatterplot in FIG. 7, the screening revealed 4 hits that inhibit PL^{pro} activity >80%: eltrombopag (DDL-701), the known PL^{pro} inhibitor GRL-0617, thrombopoietin receptor agonist-1 (TPO-1 agonist, DDL-713), and zafirlukast (DDL-715) a potent cysteinyl leukotriene receptor antagonist. Interestingly, two compounds—the antipsychotic drug fluspirilene and the leukotriene receptor antagonist montelukast—increased PL^{pro} activity.

To further validate DDL-701 as a PL^{pro} inhibitor, dose-response inhibition of PL^{pro} activity was assessed, using the PL^{pro} peptide substrate, along with the deubiquitinase and delSGylase substrates (FIG. 1) which showed IC₅₀ values of 13, 21, and 8 μM, respectively. Dose-response curves for PL^{pro} inhibition by the other hits from screening were also generated and displayed weaker potency than DDL-701: DDL-713 (IC_{50PLpro}˜54 μM) and DDL-715 (IC_{50PLpro}˜24 μM).

### DDL-701 and Nirmatrelvir (DDL-750) Show Sustained Protease Activities in Combination

For DDL-701 to be efficacious in protease inhibitor cocktail therapy, it is important that its activity be sustained in the presence of an M^{pro} inhibitor such as nirmatrelvir (DDL-750). As shown in FIG. 7A, inhibition of PL^{pro} by DDL-701 is almost complete at 50 μM and the inhibition is not affected when it is used in combination with DDL-750 in the protease assay. Similarly, DDL-750 M^{pro} inhibitory activity was sustained in the presence of DDL-701 (FIG. 7B). DDL-701 did not show activity in an M^{pro} activity assay.

### DDL-701 Activity Partially Restores Interferon-β (IFN-β) Induction In Vitro

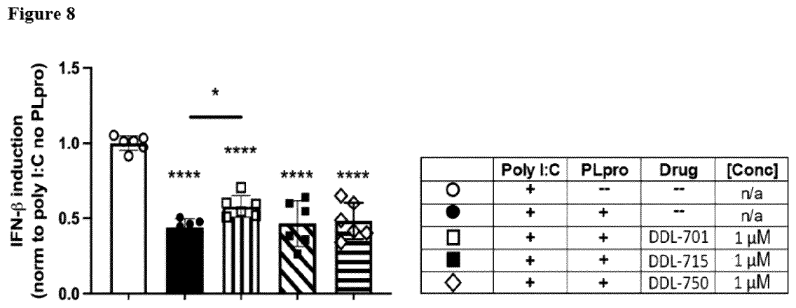

Because PL^{pro} activity is reported to inhibit the host cell IFN-β antiviral response, it was sought to determine if the PL^{pro} inhibitors can rescue IFN-β induction. HEK-293T cells were co-transfected with an IFN-β luciferase reporter and SARS-CoV-2 Wuhan strain PL^{pro}, and stimulated with polyinosinic:polycytidylic acid (poly I:C) to mimic the double stranded RNA (dsRNA) that positive-sense, single-stranded RNA viruses such as SARS-CoV-2 form during viral replication. As shown in FIG. 8, expression of PL^{pro} reduces IFN-β induction by >50%, and treatment with DDL-701 at 1 μM modestly yet significantly rescued IFN-β induction (p=0.042). Higher concentrations did not rescue induction. The lack of induction at the higher concentration could be a result of compound interference with the dual transfected HEK cell assay. Although eltrombopag is also known to target other intracellular functions that may contribute to the induction activity seen at 1 μM, however, most likely the observed induction of IFN-β in the transfected HEK cells is through inhibition of the PL^{pro} activity. Neither DDL-715 nor DDL-750 rescued IFN-β induction at any dose.

PL^{pro} activity has also been reported to antagonize NFκB signaling, therefore an NFκB assay was also performed, but no rescue of NFκB signaling was observed by any compound tested under the conditions of the assay.

### DDL-701 is Brain-Permeable in Mice

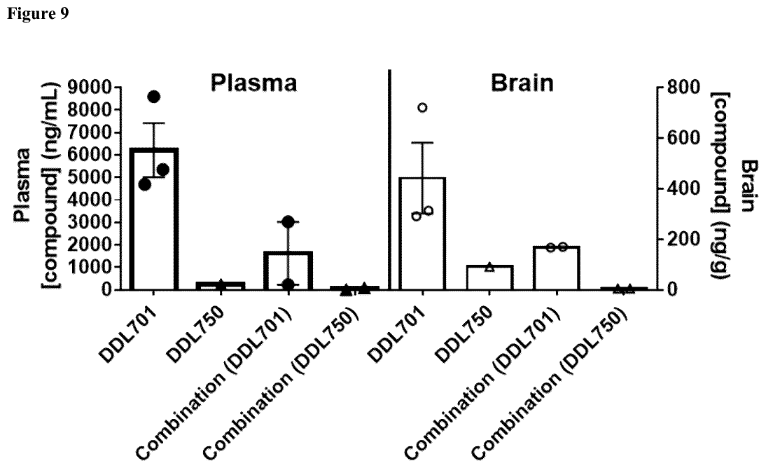

To determine if DDL-701 as part of a protease inhibitor cocktail has the potential to be effective against SARS-CoV-2 infection of the central nervous system (CNS), a pharmacokinetic study was performed in mice. Animals received DDL-701 or DDL-750 alone or in combination both at 30 mg/Kg by oral dosing, and brain and plasma levels assessed. As shown in FIG. 9, 2 hours after oral treatment, brain levels of DDL-701 were ˜1 μM (˜442 ng/g) while DDL-750 (nirmatrelvir) reached a concentration of ˜0.18 μM (˜91 ng/g). DDL-701 has >5 fold higher brain penetration than nirmatrelvir. In plasma, DDL-701 and DDL-750 levels were ˜14 1.μM (˜6,214 ng/mL) and ˜0.5 μM (˜234 ng/mL) respectively. In combination, the levels in plasma and brain were lower for both drugs; DDL-701 levels in plasma were ˜4 μM (1,628 ng/mL) and in brain −0.4 μM (˜169 ng/g), and DDL-750 levels were ˜0.1 μM (˜55 ng/mL) and ˜0.1 μM (˜59 ng/g) in plasma and brain, respectively. The drug level of DDL-701 in the mouse plasma was close to the IC₅₀ for PL^{pro} inhibition, while the levels in the brain were close to the 1 mM dose that showed IFN-β induction. In combination with DDL-750 both plasma and brain levels of DDL-701 in mice were decreased. The pilot pharmacokinetic study was done in a small group of mice and the results could be affected by mouse-to-mouse variability. The mice studies were done under an approved animal study protocol.

Modeling and Docking Reveal DDL-701 and Known PL^{pro} Iinhibitor GRL-0617 Interact with Residues Near the Active Site

Modeling by docking of DDL-701 with PL^{pro} was compared with that of the known PL^{pro} inhibitor GRL-0617 (PDB ID: 7CMD) using Cresset software. The docking data suggests that like GRL-0617, DDL-701 does not interact with the active site cysteine-111 residue, but rather can bind effectively to the site around tyrosine-268 which lies just outside the tunnel containing the active site residue Cys111. DDL-715 and the reported PL^{pro} inhibitor losartan bind the enzyme like DDL-701, at the site around Tyr268 and at the entrance of the active site tunnel leading to Cys111. Similarly, docking of DDL-713 which is a TPO agonist like DDL-701 binds around Tyr268 located outside the catalytic-site cavity containing the key residue Cys111.

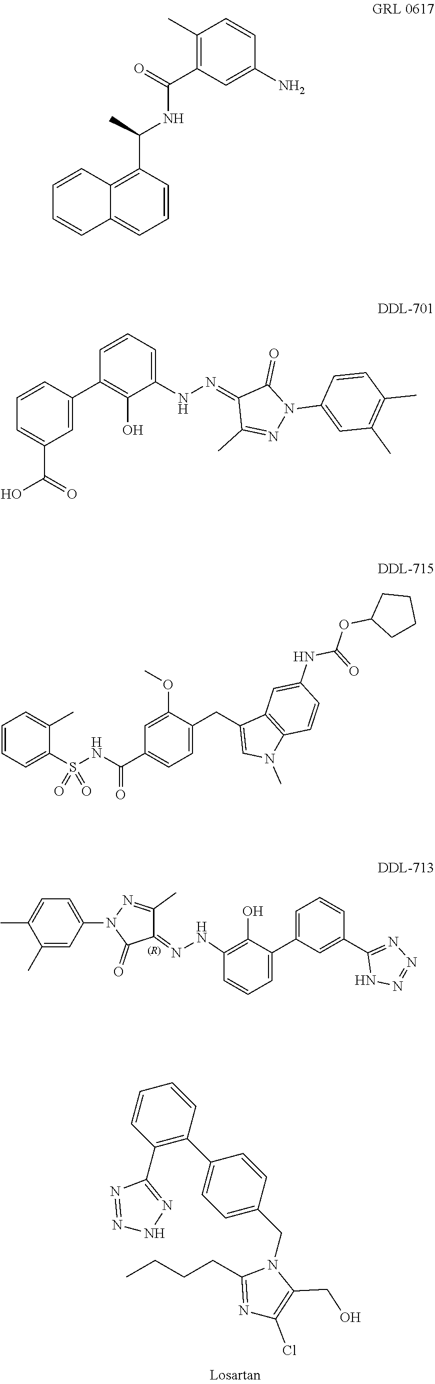

### Discussion

The FDA-approved drug DDL-701, identified in the screening of a library of clinical compounds, shows micromolar PL^{pro} inhibition and elicits partial restoration of IFN-β induction in vitro. As shown in FIGS. 7A-7B, while DDL-701 only inhibits PL^{pro} and not M^{pro} the combination protease inhibitor cocktail of DDL-701 and nirmatrelvir (DDL-750) effectively inhibits both PL^{pro} and M^{pro} enzyme activity. DDL-701 has an advantage for use in immediate clinical testing because it is already approved for thrombocytopenia (available in the US under the brand name PROMACTA and outside the US as REVOLADE), and human pharmacokinetic (PK) data is available. These reported PK data indicate that a single oral dose of 75 mg DDL-701 in tablet form results in plasma Cₘₐₓ levels in the range of 25-36 μM with an AUC of 168 μg-hour/mL, a level that is above the IC₅₀ of ˜13 μtM for inhibition of PL^{pro} enzyme. DDL-701 thus would be likely to exert PL^{pro} inhibition in vivo in human patients after an oral dose and could show robust antiviral efficacy most effectively in a protease inhibitor cocktail with a M^{pro} inhibitor.

Another PL^{pro} inhibitor identified from our screening, DDL-715, is less potent than DDL-701 and in human achieves a Cₘₐₓ of ˜0.5 μM in plasma after oral dosing. This is similar to what was shown with another PL^{pro} inhibitor losartan that also has a reported IC₅₀ of ˜13 μM for PL^{pro} inhibition. After oral dosing with losartan, however, the Cₘₐₓ in plasma is only ˜0.5 μM, well below its IC₅₀ for PL^{pro} inhibition. DDL-715 and Losartan, in our modeling, also can bind the active site region similarly to DDL-701. The PL^{pro} inhibitor tropifexor, a potent Farnesoid X Receptor agonist, is reported to have a PL^{pro} IC₅₀ ˜6 μM [25]. The binding of tropifexor in our modeling again is similar to DDL-701 around the Tyr268 site. The Cₘₐₓ in human plasma with an approved oral dose is <0.1 μM, well below its PL^{pro} IC₅₀. All of these data show that DDL-701 has an advantage, in that after a clinically used oral dose of the drug it is possible to achieve plasma levels above the PL^{pro} IC₅₀, making it a promising candidate to repurpose as part of the protease inhibitor cocktail with a M^{pro} inhibitor such as nirmatrelvir for treatment of COVID-19.

Here, the pilot PK studies in mice showing that DDL-701 is brain-permeable is also reported, which suggests that DDL-701 has potential to counter the effects of SARS-CoV-2 infection and potential complications in the CNS. Nirmatrelvir had lower brain-permeability, but it is highly potent for the M^{pro} enzyme with an IC₅₀ of ˜3 nM, therefore it may be effective in brain as part of the protease inhibitor cocktail.

As further evidence of its potential in a protease inhibitor cocktail, DDL-701 has previously been reported to have antiviral efficacy in Vero E6 cells infected with SARS-CoV-2 with an EC₅₀ of 8.2 μM and EC₉₀ of 9.5 μM, although a mechanism of action for eltrombopag in that report is not described. In Vero E6 normal cells treated with DDL-701 the CC₅₀>50 μM was reported. The reported antiviral efficacy in SARS-CoV-2 infected VeroE6 cells for nirmatrelvir was EC₅₀ of 74 nM while the reported EC₉₀ was 155 nM and in normal cells the CC₅₀ was >100 μM. In a separate study, eltrombopag was reported to show the ability to bind and reduce the stability of the spike protein-ACE2 complex, which may contribute to its antiviral efficacy. Importantly, the plasma Cₘₐₓ level of DDL-701 after a single oral dose in humans is well above this EC₅₀ for inhibition of SARS-CoV-2 infection. Thus, the oral dose of 75 mg DDL-701 as a protease inhibitor cocktail with Paxlovid in COVID-19 patients has the potential to produce robust antiviral efficacy.

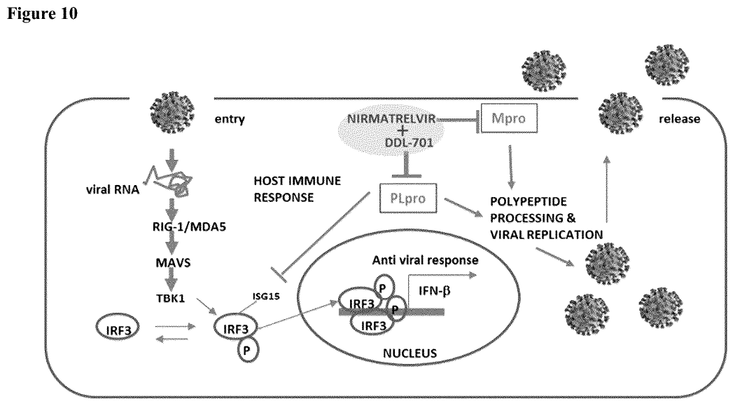

Ideally, the PL^{pro} inhibitor DDL-701 used in a protease inhibitor cocktail with the M^{pro} nirmatrelvir would result in induction of host antiviral activity, in addition to reducing viral polypeptide processing and replication. During viral infection there is induction of host antiviral response, cytosolic viral RNA forms a complex with the proteins RIG-1/MDA5, interacts with the molecular cascade involving MAVS, TBK1 and IRF3, leading to phosphorylation and nuclear translocation of IRF3 inducing transcription of type I interferons, this response can be antagonized by PL^{pro} (FIG. 10). By interfering with PL^{pro} cleavage of ISG15 and thus IRF3 phosphorylation, DDL-701 may restore IFN-β signaling in vivo, a potential effect supported by our data showing increased IFN-β induction in vitro. The lack of response to NF-_{K}B signaling could be due differential mechanisms by which its signaling is antagonized by PL^{pro}.

While the SARS-CoV-2 virus does not easily enter the brain, it has been reported to damage endothelial cells in the blood brain barrier (BBB) leading to inflammation and brain injury. Knowledge concerning the impact of SARS-CoV-2 infection on the CNS and cerebrovasculature is limited and still being elucidated. The entry of the virus into the brain through the olfactory nerve endings and at the blood-CSF barrier (choroid plexus) is postulated. Evidence also suggests that the virus can infect astrocytes in the CNS and could lead to some of the observed neurological symptoms such as fatigue, depression and brain fog. The pilot pharmacokinetic studies in mice herein suggest DDL-701 is brain-permeable, and while the brain permeability of nirmatrelvir is low, given its nanomolar potency in inhibiting viral replication as part of the protease inhibitor cocktail it may have the potential to lower the risk of CNS complications.

### Conclusions

Based on the findings in this work, DDL-701 is a PL^{pro} inhibitor with sustained inhibitory activity in the presence of M^{pro} inhibitor nirmatrelvir and has previously been reported to achieve plasma levels that are likely to elicit both PL^{pro} inhibition and antiviral efficacy. DDL-701 is a promising candidate for further clinical study in combination with a M^{pro} inhibitor as a protease inhibitor cocktail for the treatment of SARS-CoV-2 infection. DDL-701's brain permeability suggested by the studies in this work further points its potential to reduce CNS symptoms of the infection. DDL-701 has the advantage of being an FDA approved drug for thrombocytopenia and can be readily re-purposed using the already available oral tablet that could be re-formulated in combination with oral Paxlovid in the current 5-day treatment regimen. Such a protease inhibitor cocktail presents the possibility of a more effective treatment for patients worldwide with higher risk for severe COVID-19 that has the potential to reduce hospitalization, deaths resulting from the disease, speed up post-infection recovery, reduce the risk of rebound, reduce antiviral resistance and CNS complications.

### REFERENCES

Jin, Z.; Du, X.; Xu, Y.; et al., Structure of Mpro from SARS-CoV-2 and discovery of its inhibitors. Nature. 2020, 582, 289-293.

Rut, W.; Groborz, K.; et al., Substrate specificity profiling of SARS-CoV-2 main protease enables design of activity-based probes for patient-sample imaging. bioRxiv preprint. 2020, DOI:

Jeon S.; et al., Identification of antiviral drug candidates against SARS-CoV-2 from FDA-approved drugs. bioRxiv preprint. 2020, DOI: 10.1101/2020.02.20.999730.

Soneji S. Population-level mortality burden from novel coronavirus (COVID-19) in Europe and North America. Genus 2021; 77(1):7. DOI:10.1186/s41118-021-00115-9.

Li X. Omicron: Call for updated vaccines. J. Med. Virol. 2022; 94(4): 1261-1263. DOI: 10.1002/jmv.27530.

Lei J, Kusov Y & Hilgenfeld R. Nsp3 of coronaviruses: Structures and functions of a large multi-domain protein. Antiviral Res. 2018; 149, 58-74. DOI: 10.1016/j.antiviral.2017.11.001

Ratia K et al. Structural Basis for the Ubiquitin-Linkage Specificity and deISGylating Activity of SARS-CoV Papain-Like Protease. PLoS Pathog. 2014; 10, e1004113. DOI: 10.1371/journal.ppat.1004113

Yadav R et al., Role of Structural and Non-Structural proteins and therapeutic targets and SARS-CoV-2 for COVID-19. Cell 2021; 10(4):821. DOI: 10.3390/cells10040821.

Shin D. et al. Papain-like protease regulates SARS-CoV-2 viral spread and innate immunity. Nature 2020; 587, 657-662. DOI: 10.1038/s41586-020-2601-5.

Matthews K., et al. The SARS coronavirus papain-like protease can inhibit IRF3 at a post activation step that requires deubiquitination activity. Virol. J. 2014; 11, 209. DOI: 10.1186/s 12985-014-0209-9.

Freitas B, et al. Characterization and Noncovalent Inhibition of the Deubiquitinase and deISGylase Activity of SARS-CoV-2 Papain-Like Protease. ACS Infect. Dis. 2020; 6, 8, 2099-2109. DOI: 10.1021/a csinfecdis.0c00168.

Li C, et al. Viral Macro Domains Reverse Protein ADP-Ribosylation. J. Virol. 2016; 90, 8478-8486. DOI: 10.11285V1.00705-16.

Malgras M, et al. The Antiviral Activities of Poly-ADP-Ribose Polymerases. Viruses 2021; 13, 582. DOI: 10.3390/v13040582.

Sun L, et al. Coronavirus Papain-like Proteases Negatively Regulate Antiviral Innate Immune Response through Disruption of STING-Mediated Signaling. PLoS ONE 2012; 7, e30802. DOI: 10.1371/journal.pone.0030802.

Mielech A M, et al. MERS-CoV papain-like protease has deISGylating and deubiquitinating activities. Virology 2014; 450-451, 64-70. DOI: 10.1016/j.viro1.2013.11.040.

Ma-Lauer Y, et al. p53 down-regulates SARS coronavirus replication and is targeted by the SARS-unique domain and PL pro via E3 ubiquitin ligase RCHY1. (2016) Proc Natl Acad Sci USA. 2016 Aug. 30; 113(35):E5192-201. DOI: 10.1073/pnas.1603435113.

Hadjadj J, et al. Impaired type I interferon activity and inflammatory responses in severe COVID-19 patients. Science 2020; 369, 718-724. DOI: 10.1126/science.abc6027.

Owen D R, et al. An oral SARS-CoV-2 Mpro inhibitor clinical candidate for the treatment of COVID-19. Science 2021; 374(6575):1586-1593. DOI: 10.1126/science.ab14784.

Gupta K, et al. Rapid relapse of symptomatic omicron SARS CoV-2 infection following early suppression with nirmatrelvir/ritonavir. 26 Apr. 2022, PREPRINT (Version 1) available at Research Square [https://doi.org/10.21203/rs.3.rs-1588371/v1].

Carlin A F, et al. Virologic and Immunologic Characterization of COVID-19 Recrudescence after Nirmatrelvir/Ritonavir Treatment. 18 May 2022, PREPRINT (Version 1) available at Research Square [https://doi.org/10.21203/rs.3.rs-1662783/v1].

Sacco M D, et al. The P132H mutation in main protease of omicron SARS-CoV-2 decreases thermal stability without compromising catalysis or small-molecule drug inhibition. Cell Res 2021; 32, 498-500. DOI: 0.1038/s41422-022-00640-y.

Zhou Y, et al., Nirmatrelvir Resistant SARS-CoV-2 Variants with High Fitness In vitro. BioRxiv 2022; DOI:.10.1101/2022.06.06.494921.

Jeon S, et al. Identification of Antiviral Drug Candidates against SARS-CoV-2 from FDA-Approved Drugs. Antimicrob Agents Chemother. 2020; 64(7): e00819-20. DOI: 10.1128/AAC.00819-20.

Nejat R et al., Losartan inhibits SARS-CoV-2 Replication in vitro. J. Pharm Pharm Sci. 2021; 24, 390-399. DOI: 10.18433/jpps31931.

Jamir E, et al. Applying polypharmacology approach for drug repurposing for SARS-CoV-2. J. Chem. Sci. 2022, 134;57. DOI: 10.1007/s12039-022-02046-0.

Lewis D S M et al., Aloin isoforms (A and B) selectively inhibits proteolytic and deubiquinating activity of PLpro of SARS-CoV-2 in vitro. Sci. Rep. 2022; 12, 2145. DOI: 10.1038/s41598-022-06104-y.

Napolitano V, et al., Acriflavine, a clinically approved drug, inhibits SARS-CoV-2 and other betacoronaviruses. Cell Chem Bio 2011; 29, 774-784. DOI: 10.1016/j.chembiol.2021.11.006.

Chunlong M, et al., Drug-Repurposing Screening Identified Tropifexor as a SARS-CoV-2 Papain-like Protease Inhibitor. ACS Infectious Disease 2022; 8, 1022. DOI: 10.1021/acsinfeedis.1c00629.

Narayanan A, et. Al., Identification of SARS-CoV-2 inhibitors targeting Mpro and PLpro using in cell protease assay. Commun. Biol. 2022; 5, 169. DOI: 10.1038/s42003-022-03090-9.

Kulandaisamy R, et al., Repurposing of FDA approved Drugs against SARS-CoV-2 papain-like protease: Computational, Biochemical and in vitro studies. Front. Microbiol. 2022; 13:877813. DOI: 10.3389/fmicb.2022.877813.

Zhang L, et al., Crystal structure of SARS-CoV-2 main protease provides a basis for design of improved a-ketoamide inhibitors. Science 2020; 368(6489): 409-412. DOI: 10.1126/science.abb3405.

Mellott D M, et al., A clinical-stage cysteine protease inhibitor blocks SARS-CoV-2 infection of Human and Monkey Cells. ACS Chem. Biol. 2021, 16, 642-650. DOI: 10.1021/acschembio.0c00875.

Fu Z et al. The complex structure of GRL0617 and SARS-CoV-2 PLpro reveals a hot spot for antiviral drug discovery. Nat Commun. 2021;12: 488. DOI: 10.1038/s41467-020-20718-8.

Murata Y & Sugimoto O. Zafirlukast (Accolate): a review of its pharmacological and clinical profile. Nihon Yakurigaku Zasshi. 2002 April;119(4):247-58. DOI: 10.1254/fpj.119.247.

Guan Y et al., Eltrombopag inhibits TET dioxygenase to contribute to hematopoietic stem cell expansion in aplastic anemia. J. Clin Invest. 2022 132(4):e149856. DOI.org/10.1172/JCI149856.

Frieman M, et al., Severe acute respiratory syndrome coronavirus papain-like protease ubiquitin-like domain and catalytic domain regulate antagonism of IRF3 and NF-kappaB signaling. J. Virol. 2009;83(13):6689-705. DOI: 10.1128/JVI.02220-08.

Gao X, et al., Crystal structure of SARS-CoV-2 papain-like protease. Acta Pharm Sin B. 2021; 11: 237-245. DOI: 10.1016/j.apsb.2020.08.014.

Matthys et al., Clinical pharmacokinetics, platelet response, and safety of eltrombopag at supratherapeutic doses of up to 200 mg once daily in healthy volunteers. J Clin Pharmacol. 2011 March;51(3):301-8. DOI: 10.1177/0091270010368677.

Gupta, M and Weaver D. COVID-19 as a trigger of Brain Autoimmunity. ACS Chem Neurosci. 2021; 12:2558. DOI: 10.1021/acschemneuro.1c00403.

Feng S et al. Eltrombopag is a potential target for drug intervention in SARA-CoV-2 spike protein. Infect Genet Evol. 2020, 85: 104419. DOI: 10.1016/j.meegid.2020.104419.

GuanQun L, et al., ISG15-dependent activation of the sensor MDAS is antagonized by the SARS-CoV-2 papain-like protease to evade host innate immunity. Nat Microbiol. 2021, 6: 467-468. DOI: 10.1038/s41564-021-00884-1.

Brisse M., et al., Comparative Structure and function analysis of the RIG-1-like receptors: RIG-1 and MDAS. Front. Immunol. 2019, 10: 1586. DOI: 10.3389/fimmu.2019.01586.

Boldrini M, et al. How COVID-19 affects the brain. JAMA Psychiatry 2021;78(6):682-683. DOI: 10.1001/jamapsychiatry.2021.0500.

McQuaid C, et al., SARS-Cov-2: is there neuroinvasion? Fluids Barriers CNS 2021; 18: 32. DOI: 10.1186/s12987-021-00267-y.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating a coronavirus infection comprising conjointly administering to a subject in need thereof:
a PLpro inhibitor, or a pharmaceutically acceptable salt or prodrug thereof; and
an Mpro inhibitor, or a pharmaceutically acceptable salt or prodrug thereof.

2. The method of claim 1, wherein the coronavirus is SARS-CoV-2.

3. The method of claim 1, wherein the PLpro inhibitor is: 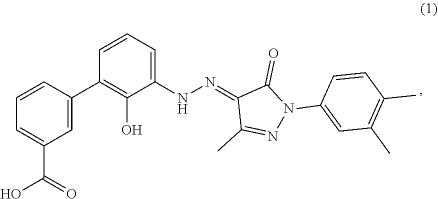 [SMILES: CC1=NN(c2ccc(C)c(C)c2)C(=O)/C1=N/Nc1cccc(-c2cccc(C(=O)O)c2)c1O]
or a pharmaceutically acceptable salt thereof.

4. The method of claim 1, wherein the PLpro inhibitor is: 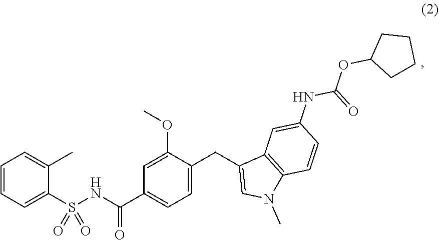 [SMILES: COc1cc(C(=O)NS(=O)(=O)c2ccccc2C)ccc1Cc1cn(C)c2ccc(NC(=O)OC3CCCC3)cc12]
or a pharmaceutically acceptable salt thereof.

5. The method of claim 1, wherein the Mpro inhibitor is: 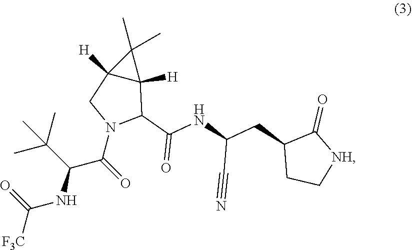 [SMILES: CC(C)(C)[C@H](NC(=O)C(F)(F)F)C(=O)N1C[C@H]2[C@@H](C1C(=O)N[C@H](C#N)C[C@@H]1CCNC1=O)C2(C)C]
or a pharmaceutically acceptable salt thereof.

6. The method of claim 1, further comprising administering ritonavir: 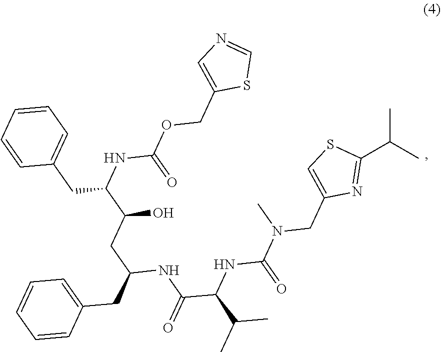 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]
or a pharmaceutically acceptable salt thereof.

7. The method claim 1, wherein the steady state plasma Cₘₐₓ of the PLpro inhibitor is at least about 10 μM.

8. (canceled)

9. (canceled)

10. The method of claim 1, wherein the PLpro inhibitor is administered orally.

11. The method of claim 1, wherein the Mpro inhibitor is administered orally.

12. (canceled)

13. (canceled)

14. The method of claim 1, wherein the PLpro inhibitor and an Mpro inhibitor are administered concomitantly.

15. The method of claim 1, wherein the PLpro inhibitor and an Mpro inhibitor are administered sequentially.

16. The method of claim 1, wherein the PLpro inhibitor is administered in a first pharmaceutical composition that comprises one or more pharmaceutically acceptable excipients.

17. The method of claim 1, wherein the Mpro inhibitor is administered in a second pharmaceutical composition that comprises one or more pharmaceutically acceptable excipients.

18.-21. (canceled)

22. The method of any one of claims 16, wherein the first composition comprises between 50-100 mg of the PLpro inhibitor.

23. The method of claim 22, wherein the first composition comprises about 75 mg of the PLpro inhibitor.

24. A pharmaceutical composition comprising:
a PLpro inhibitor, or a pharmaceutically acceptable salt or prodrug thereof;
an Mpro inhibitor, or a pharmaceutically acceptable salt or prodrug thereof; and
one or more pharmaceutically acceptable excipients.

25. The pharmaceutical composition of claim 24, wherein the PLpro inhibitor is: 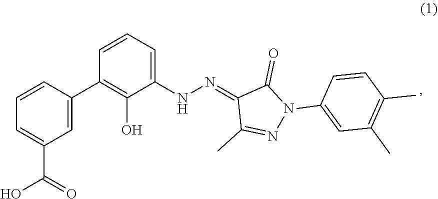 [SMILES: CC1=NN(c2ccc(C)c(C)c2)C(=O)/C1=N/Nc1cccc(-c2cccc(C(=O)O)c2)c1O]
or a pharmaceutically acceptable salt thereof.

26. The pharmaceutical composition of claim 24, wherein the PLpro inhibitor is: 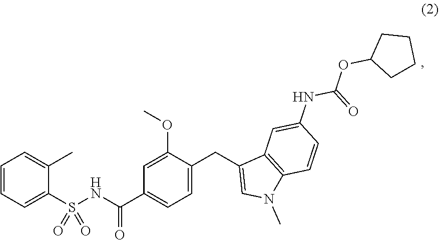 [SMILES: COc1cc(C(=O)NS(=O)(=O)c2ccccc2C)ccc1Cc1cn(C)c2ccc(NC(=O)OC3CCCC3)cc12]
or a pharmaceutically acceptable salt thereof.

27. The pharmaceutical composition of claim 24, wherein the Mpro inhibitor is: 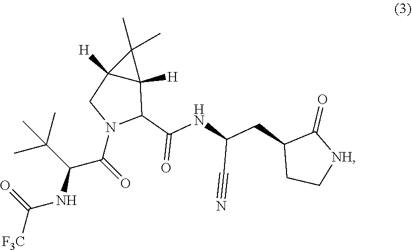 [SMILES: CC(C)(C)[C@H](NC(=O)C(F)(F)F)C(=O)N1C[C@H]2[C@@H](C1C(=O)N[C@H](C#N)C[C@@H]1CCNC1=O)C2(C)C]
or a pharmaceutically acceptable salt thereof.

28. The pharmaceutical composition of claim 24, further comprising ritonavir: 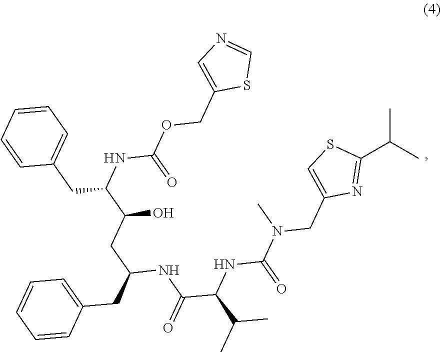 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]
or a pharmaceutically acceptable salt thereof.
